(19) United States
(12) Patent Application Publication
(10) Pub. No.: US 2022/0176008 A1
(43) Pub. Date: Jun. 9, 2022

(54) Medical Fixing Material with Outstanding Breathability and Ease of Application

(71) Applicant: SANOVITA CO., LTD., Tokyo (JP)

(72) Inventor: Mihoro SANO, Tokyo (JP)

(21) Appl. No.: 17/605,229

(22) PCT Filed: Feb. 10, 2020
(86) PCT No.: PCT/JP2020/005200

§ 371 Date: Oct. 20, 2021

(30) Foreign Application Priority Data
Apr. 21, 2019 (JP) 2019-080581

(51) Int. Cl. A61L 15/14; A61L 15/12; A61F 13/04; A61F 5/058

(52) U.S. Cl. CPC A61L 15/14; A61F 5/058; A61F 13/04; A61L 15/12

(57) ABSTRACT

To provide a surgical fixing material which has excellent breathability and ease of application, and which is not susceptible to causing rigid wrinkles when fixing bent sites. A surgical fixing material having a hardened layer 5 which comprises a plurality of holes 3 having a surface area of 5 mm² or more.

### TECHNICAL FIELD

The invention relates to a fixing material having a plurality of holes, and a plaster bandage and a splint including the fixing material.

### BACKGROUND ART

JP-A-2-268760 discloses a surgical plaster bandage. Furthermore, JP-A-2015-33554 discloses a simple splint. The splint and the plaster bandage are medical tools used for fixing a target site. Meanwhile, when wearing the splint or the plaster bandage, there has been a problem with breathability, and a problem with the splint or the plaster bandage hardening in a wrinkled state.

Patent Document 1: JP-A-2-268760

Patent Document 2: JP-A-2015-33554

### DISCLOSURE OF THE INVENTION

### Problems to be Solved by the Invention

An object of the invention disclosed in the description is to provide a splint and a plaster bandage with excellent breathability and ease of application, and a fixing material used for the splint and the plaster bandage. Particularly, an object of the invention is to provide a roll shaped surgical fixing material that is not susceptible to causing rigid wrinkles when fixing a bent site.

### Solutions to the Problems

One of the inventions disclosed in the description relates to a surgical fixing material 1. The surgical fixing material 1 includes a hardened layer 5 having a plurality of holes 3.

The surgical fixing material 1 may be a plaster bandage, or a fixing material for a splint.

Each of the plurality of holes preferably has a surface area of 5 mm² or more.

The plurality of holes may have centers existing on lattice points of a square lattice, a rectangular lattice, a face-centered rectangular lattice, an orthorhombic lattice, or a hexagonal lattice. The plurality of holes may have centers existing randomly.

The hardened layer may have two frame portions extending in a longitudinal direction of the surgical fixing material and a plurality of connecting portions that connect the two frame portions.

When the surgical fixing material 1 is the fixing material for a splint, the splint has two covering layers that cover the fixing material from both surfaces. These covering layers also have a plurality of holes that preferably communicate with the holes of the hardened layer 5. That is, the splint has a first covering layer that covers a first surface of the surgical fixing material, and a second covering layer that covers a second surface of the surgical fixing material, and the first covering layer and the second covering layer have a plurality of holes of the covering layers that connect to the respective plurality of holes.

### Advantageous Effects of the Invention

According to the invention, since the hardened layer has the plurality of holes, a splint and a plaster bandage with excellent breathability and ease of application, and a fixing material therefor can be provided.

### BRIEF DESCRIPTION OF THE DRAWINGS

FIG. 1 is a conceptual diagram for describing a surgical fixing material.

FIG. 2 is a conceptual diagram illustrating exemplary configurations of a plurality of holes.

FIG. 3 is a conceptual diagram illustrating examples of a surgical fixing material with dotted lines.

FIG. 4 is a conceptual diagram for describing an example of the surgical fixing material in which a hardened layer has two frame portions and a plurality of connecting units that connect the two frame portions.

FIG. 5 is a diagram for describing an example of a splint having a plurality of holes in the hardened layer.

### DESCRIPTION OF PREFERRED EMBODIMENTS

The following describes an embodiment for carrying out the present invention using the drawings. The present invention is not limited to the embodiment described below and includes ones appropriately modified in an obvious range by those skilled in the art from the following embodiment.

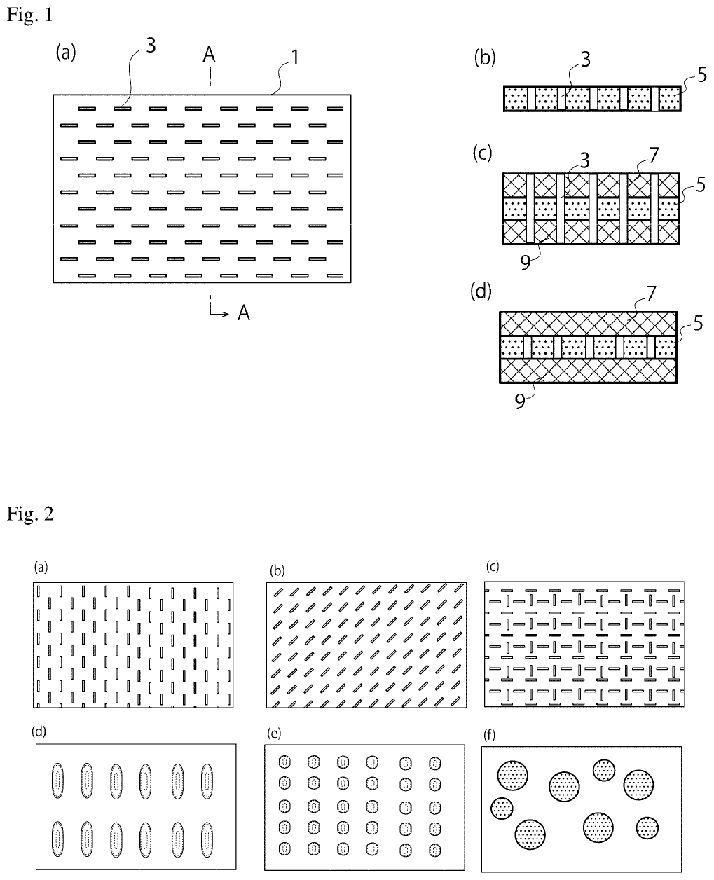

FIG. 1 is a conceptual diagram for describing the surgical fixing material. FIG. 1(a) is a front view. FIG. 1(b) to FIG. 1(d) illustrate the A-A cross-sectional views of FIG. 1(a). FIG. 1(b) illustrates a surgical fixing material configured only by a hardened layer. FIG. 1(c) illustrates a surgical fixing material configured by the hardened layer (core material layer) and two covering layers and having holes that penetrate through the core material layer and the covering layers. FIG. 1(d) illustrates a surgical fixing material configured by the hardened layer (core material layer) and the two covering layers and having holes that exist in the core material layer.

The surgical fixing material 1 is a material that has a flexibility before surgery and hardens after surgery to fix a target site. The surgical fixing material is used for fixing a broken bone, an injured joint, or a sprain. The surgical fixing material 1 may be a plaster bandage, or a fixing material for a splint. The surgical fixing material 1 may be roll shaped, or preliminarily be shaped suitable for the shape of the target site. The invention can be particularly preferable for being used with a roll shaped surgical fixing material.

As illustrated in FIG. 1, the surgical fixing material 1 includes a hardened layer 5 having a plurality of holes 3. An example of the hardened layer 5 contains a water-hardening resin. The water-hardening resin gradually hardens by absorbing water. The hardened layer contains a fixing material and is also referred to as the core material layer. The fixing material hardens by causing a reaction with water, and thus becomes capable of covering and fixing the affected part. An example of the hardened layer includes polyester, glass fiber, or polyurethane.

Each of the plurality of holes preferably has a surface area of 5 mm² or more. Each of the plurality of holes may have a surface area of 0.1 cm² or more and 15 cm² or less, 0.4 cm² or more and 10 cm² or less, or 0.5 cm² or more and 5 cm² or less. The total surface area of the plurality of holes may be 1% or more and 99% or less, 5% or more and 50% or less, or 80% or more and 99% or less of a surface area of one side surface of the hardened layer. Since the surgical fixing material has such holes, even when fixing a joint, for example, the surgical fixing material can be shaped according to the shape of a motion range without causing wrinkles. When using a roll shaped splint to fix a joint or the like, an excessive portion of the splint occurs, resulting in the solidified splint in a wrinkled state. Therefore, in some cases, the rigid wrinkles hurt surroundings thereof or hurt the patient him/herself. Since the surgical fixing material has a plurality of holes in the area described above, even when the roll shaped splint is used in a bent site, the holes absorb the excessive portion and allows effectively suppressing the cause of wrinkles.

FIG. 2 is a conceptual diagram illustrating exemplary configurations of a plurality of holes. FIG. 2(a) illustrates a configuration provided with a plurality of holes having a long axis in a perpendicular direction with respect to the longitudinal direction (long direction) of the roll shaped surgical fixing material. FIG. 2(b) illustrates a configuration provided with a plurality of holes having a long axis in an inclined direction with respect to the longitudinal direction of the roll shaped surgical fixing material. FIG. 2(c) illustrates a configuration provided with a plurality of holes having a long axis in the perpendicular direction with respect to the longitudinal direction of the roll shaped surgical fixing material, and further provided with a plurality of holes having a long axis in a parallel direction to the longitudinal direction. FIG. 2(d) illustrates a configuration in which a plurality of holes are circular. FIG. 2(e) illustrates a configuration in which a plurality of holes are ellipsoid. FIG. 2(f) illustrates a configuration in which a plurality of holes in various sizes exist randomly. In the example of FIG. 1(a), a plurality of holes having a long axis in the parallel direction with respect to the longitudinal direction of the roll shaped surgical fixing material are disposed. Meanwhile, the configuration of FIG. 2(a) is provided with the plurality of holes having the long axis in the perpendicular direction with respect to the longitudinal direction. The direction of the holes allows an extending direction of the surgical fixing material before solidifying to be adjusted. A surgical fixing material having appropriate holes may be selected according to a bending degree of the surgical fixing material used in the applied site. The configuration in FIG. 2(f) has an advantage of having an excellent design, maintaining functionality while also being visually not boring.

The plurality of holes may have centers existing on the lattice points of the square lattice, the rectangular lattice, the face-centered rectangular lattice, the orthorhombic lattice, or the hexagonal lattice. The plurality of holes may have centers existing randomly.

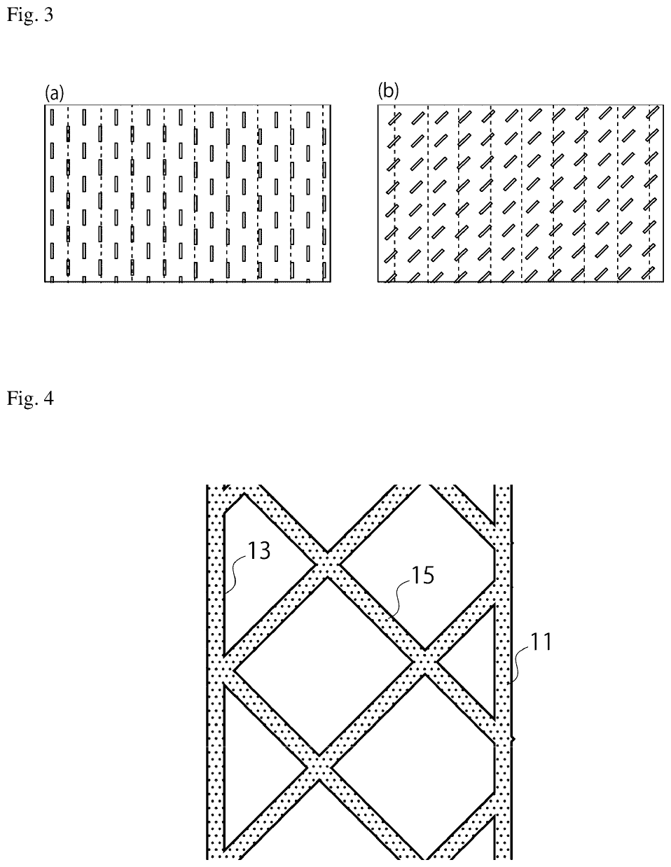

FIG. 3 is a conceptual diagram illustrating examples of the surgical fixing material having dotted lines. FIG. 3(a) illustrates a surgical fixing material in which the dotted lines are disposed so as to connect neighboring holes among the plurality of holes. FIG. 3(b) illustrates an example of a surgical fixing material in which the dotted lines are disposed irrespectively of the positions of the holes. The dotted lines are illustrated as dashed lines in the diagram. A user can cut the surgical fixing material along the dashed lines with scissors, for example. Accordingly, size of the holes can be easily adjusted according to the target site.

In a case where the surgical fixing material 1 is a fixing material for a splint, the splint preferably has two covering layers that cover the fixing material from both surfaces. The covering layers also have a plurality of holes that preferably communicate with the holes of the hardened layer 5. That is, the splint has a first covering layer 7 covering a first surface of the surgical fixing material, and a second covering layer 9 covering a second surface of the surgical fixing material, and the first covering layer and the second covering layer have a plurality of holes 11. 13 of the covering layers that connect to the respective plurality of holes. The covering layer may be configured by a nonwoven fabric, for example. An example of the material of the covering layer is polypropylene.

FIG. 4 is a conceptual diagram for describing an example of a surgical fixing material in which a hardened layer has two frame portions and a plurality of connecting units that connect the two frame portions. As illustrated in FIG. 4, the hardened layer may have two frame portions 11. 13 extending in the longitudinal direction of the surgical fixing material, and a plurality of connecting portions 15 connecting the two frame portions. In this case, portions surrounded by the frame portions and the connecting units are equivalent to the holes.

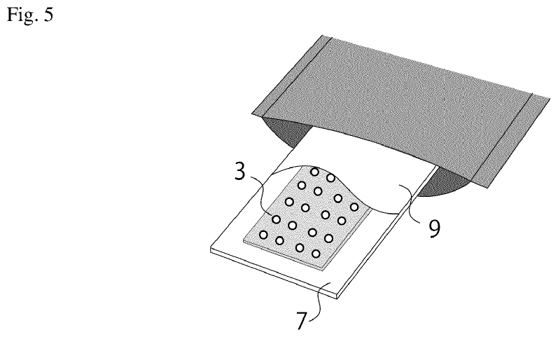

FIG. 5 is a diagram for describing an example of a splint having a plurality of holes in the hardened layer. In this case, the splint is housed in an aluminum or the like, such that the splint does not absorb water.

The above-described fixing material is immersed in water, for example, at the time of use. The fixing material containing water is wound to the target site. Accordingly, the hardened layer included in the fixing material hardens and allows the fixing material to fix the target site.

### INDUSTRIAL APPLICABILITY

Since the invention relates to a surgical splint, a plaster bandage, and the like, the invention may be used in a medical equipment industry.

### DESCRIPTION OF REFERENCE SIGNS

1 . . . surgical fixing material
3. . . hole
5. . . hardened layer (core material layer)
7. . . first covering layer
9. . . second covering layer
11, 13. . . frame
15. . . connecting portion

1. A surgical fixing material comprising
a hardened layer 5 having a plurality of holes 3.

2. The surgical fixing material according to claim 1, wherein
each of the plurality of holes has a surface area of 5 mm² or more.

3. The surgical fixing material according to claim 2, wherein
the surgical fixing material is a plaster bandage, or a fixing material for a splint.

4. The surgical fixing material according to claim 3, wherein
the plurality of holes have centers existing on lattice points of a square lattice, a rectangular lattice, a face-centered rectangular lattice, an orthorhombic lattice, or a hexagonal lattice, or existing randomly.

5. The surgical fixing material according to claim 2, wherein
the hardened layer has two frame portions extending in a longitudinal direction of the surgical fixing material and a plurality of connecting portions that connect the two frame portions.

6. A splint comprising:
the surgical fixing material according to claim 1;
a first covering layer that covers a first surface of the surgical fixing material; and
a second covering layer that covers a second surface of the surgical fixing material, wherein
the first covering layer and the second covering layer have a plurality of holes of the covering layers that connect to the respective plurality of holes.
